(19) United States
(12) Patent Application Publication
(10) Pub. No.: US 2010/0021924 A1
(43) Pub. Date: Jan. 28, 2010

(54) Transgenic animal and methods for decreasing cardiac cell death via cardiac-specific sir2alpha overexpression

(76) Inventor: Junichi Sadoshima, Berkeley, NJ (US)

Correspondence Address: Jane Massey Licata;Licata & Tyrrell P.C., 66 E. Main Street, Marlton, NJ, 08053, US

(21) Appl. No.: 12/575,021

(22) Filed: Oct. 7, 2009

Related U.S. Application Data
(63) Continuation of application No. 11/548,903, filed on Oct. 12, 2006.
(60) Provisional application No. 60/726,259, filed on Oct. 13, 2005.

(51) Int. Cl. C12Q 1/68

(52) U.S. Cl. 435/6

(57) ABSTRACT

The present invention relates to a transgenic animal and methods for increasing the expression or activity of Sir2α protein. An increase in the expression of Sir2α protein prevents stress- and age-related cardiac cell death thereby facilitating the treatment of cardiac diseases or conditions associated with aging.

### INTRODUCTION

This application is a continuation of U.S. Ser. No. 11/548,903 filed Oct. 12, 2006, which claims benefit of U.S. Provisional Patent Application Ser. No. 60/726,259, filed Oct. 13, 2005, each of which are herein incorporated by reference in their entireties.

### BACKGROUND OF THE INVENTION

Extrinsic and intrinsic factors cooperate in determining the rate of aging and the aging phenotype. Growing lines of evidence suggest that the life-span of organisms is regulated by defined molecular mechanisms (Kenyon (2001) Cell 105:165-168). These include silent information regulator2 (Sir2) family histone deacetylases (HDACs); anti-oxidants, such as superoxide dismutase (SOD), catalase and thioredoxin (Trx); Forkhead box class O (FOXO) family transcription factors; and adenylyl cyclase type 5 (AC5)/protein kinase A (PKA-dependent signaling mechanisms. These mechanisms are evolutionarily conserved and thus expected to regulate fundamental biological functions. In fact, several mechanisms causing life-span extension in yeast and nematodes induce life-span extension in mammals, including mice and primate as well. These factors not only extend the maximum life-span, but also retard the aging process in a wide variety of animals. Importantly, these longevity factors in the lower organisms usually also confer stress resistance to the organism (Kenyon (2001) supra; Longo (1999) Neurobiol Aging 20:479-486; Longo and Fabrizio (2002) Cell Mol Life Sci. 59:903-908).

Evolutionarily conserved defined molecular mechanisms may be involved in the regulation of life-span in animals (Finch and Ruvkun (2001) supra; Martin and Friedman (2004) Sci. Aging Knowledge Environ. 2004:pe32; Tissenbaum and Guarente (2002) Dev. Cell. 2:9-19; Quarrie and Riabowol (2004) Sci. Aging Knowledge Environ. 2004:re5). Among them, caloric restriction consistently prolongs life-span of organisms, from yeast to primates (Koubova and Guarente (2003) Genes Dev. 17:313-321), possibly lowering the rate of free radical formation (Sohal and Weindruch (1996) Science 273:59-63) and/or activating Sir2 or Hst2, a Sir2 homolog, which increases transcriptional silencing and promotes the stability of repetitive ribosomal RNA (Cohen, et al. (2004) Science 305:390-392; Lin, et al. (2002) Nature 418:344-348; Lamming, et al. (2005) Science 309:1861-1864). Genetic alterations causing metabolic effects similar to caloric restriction, including mutation in AC, PIL4 and deletion of hexokinase, also cause life-span extension in yeast. Of significance, caloric restriction reduces age-related pathologies and diseases in animals (Turturro, et al. (2002) J. Gerontol. A Biol. Sci. Med. Sci. 57:8379-389).

The increase in longevity by caloric restriction is analogous to that caused by defects in the neuroendocrine system in many respects (Sohal and Weindruch (1996) supra). For example, inhibition of insulin-like growth factor (IGF-I) signaling, including Daf2 mutation in C elegans, also causes life-span extension (Kimura, et al. (1997) Science 277:942-946), mediated by loss of suppression of Dafl6 or FOXO transcription factors, which regulate expression of anti-oxidants (MnSOD) and DNA damage repair (GADD45) (Murphy, et al. (2003) Nature 424:277-283; Honda and Honda (1999) FASEB J. 13:1385-1393; Fabrizio, et al. (2001) Science 292:288-290; Kops, et al. (2002) Nature 419:316-321). These mechanisms are also known to regulate dauer formation in C elegans. In mammals, Ames and Snell dwarf mice lacking GH/IGF-I signaling and IGF-I receptor heterozygous knockout mice (Holzenberger. et al. (2003) Nature 421:182-187) have longer life-spans (Tatar, et al. (2003) Science 299:1346-1351). Systemic overexpression of klotho, a hormone known to inhibit insulin/IGF-I signaling extends life-span in mice (Kurosu, et al. (2005) Science 309:1829-1833). By contrast, although klotho^{-/-} mice develop multiple age-related disorders and die prematurely, a genetic cross with mice heterozygous for an IRS-1 null allele partially ameliorates age-related phenotype and short life-span (Kurosu, et al. (2005) supra). However, whether or not inhibiting IGF-I signaling (and thus stimulating FOXOs) positively affects aging and aging-related diseases in mammals without affecting normal function has not been appreciated (Crow (2004) Circ. Res. 95:953-956). IGF-I may only produce a trade-off between current benefits to reproduction and later costs in senescence, like the relationship between inotropic agents and exacerbation of heart failure (Anversa (2005) Circ. Res. 97:411-414).

Several downstream molecules are commonly involved in life-span extension. Among these, the Sir2 family of class III HDACs plays an essential role in mediating life-span extension/cell survival in response to caloric restriction (Cohen, et al. (2004) Science 305:390-392; Lin, et al. (2000) Science 289:2126-2128; Rogina and Helfand (2004) Proc. Natl. Acad. Sci. USA 101:15998-16003). In yeast, hyperosmotic stress also extends life-span through Sir2-dependent mechanisms (Kaeberlein, et al. (2002) Mol. Cell Biol. 22:8056-8066). Overexpression of Sir2 increases the life-span of many organisms, including yeast, C. elegans, Drosophia (Rogina and Helfand (2004) supra; Wood, et al. (2004) Nature 430:686-689), and mammals and has been suggested as a target for increasing life-span (U.S. patent application Ser. No. 10/191,121). The effect of Sir2α on life-span extension is mediated by multiple mechanisms, including rDNA silencing and deacetylation of FOXO (Daitoku, et al. (2004) Proc. Natl. Acad. Sci. USA 101:10042-10047). Another important mechanism controlling aging is oxidative stress (Sohal and Weindruch (1996) supra). Overexpression of antioxidant molecules, including SODs, catalase, and thioredoxin (Mitsui, et al. (2002) Antioxid. Redox Signal 4:693-696; Murata, et al. (2002) Mol. Immunol. 38:747-757; Taub, et al. (1999) Nature 399:162-166), induces life-span extension. p66^{shc}-null mice have a 30% longer life-span and exhibit reduced apoptosis in response to oxidative stress (Migliaccio, et al. (1999) Nature 402:309-313). In these mice, FOXOs have higher levels of activity and upregulates antioxidant genes (Nemoto and Finkel (2002) Science 295:2450-2452). Besides these mechanisms, several other mechanisms, including TOR (as a potential sensor to detect nutrients) (Vellai, et al. (2003) Nature 426:620; Kapahi, et al. (2004) Curr. Biol. 14:885-890), 4E-BP1 (Tettweiler, et al. (2005) Genes Dev. 19:1840-1843), HSF-1 (Hsu, et al. (2003) Science 300:1142-1145) and AMPK (Apfeld, et al. (2004) Genes Dev. 18:3004-3009), have been shown to be important in determining the life-span of various organisms. However, it is unclear whether or not these regulators of longevity also affect aging and the stress resistance of individual organs.

Aging hearts exhibit unique histological and biochemical features (Sussman, et al. (2004) Annu. Rev Physiol. 66:29-48; Lakatta (2003) Circulation 107:490-497; Lakatta and Levy (2003) Circulation 107:139-146; Lakatta and Levy (2003) Circulation 107:346-354). Namely, increases in apoptosis and necrosis, proliferation of myocyte nuclei, increased myocyte volume, and connective tissue accumulation are frequently observed in the myocardium of old animals (Kajstura, et al. (1996) Am. J. Physiol. 271:H1215-1228; Swynghedauw, et al. (1995) Am. J. Cardiol. 76:2D-7D; Olivetti, et al. (1994) J. Am. Coll. Cardiol. 24:140-149). Changes in molecular expression commonly seen in aging hearts include modulation of p16^{INK4} and p19^{ARF} (Krishnamurthy, et al. (2004) J. Clin. Invest. 114:1299-1307; Satyanarayana and Rudolph (2004) J. Clin. Invest. 114:1237-1240). Aging cardiac myocytes are incapable of hypertrophy and proliferation, express cell cycle inhibitors, and cannot be transcriptionally reprogrammed in response to increased workload (Sussman, et al. (2004) supra; Kajstura, et al. (2000) Am. J. Pathol. 156:813-819; Takahashi, et al. (1992) J. Clin. Invest. 89:939-946; Nadal-Ginard, et al. (2003) Circ. Res. 92:139-150; Levine, et al. (2002) Am. J. Geriatr. Cardiol. 11:299-304). Aging increases wall stress that is not normalized by ventricular remodeling (Capasso, et al. (1990) Am. J. Physiol. 259:H1086-1096). Induction of cell protective mechanisms, such as expression of antioxidant and heat shock proteins, in response to pathologic insults is attenuated in aging hearts (Takahashi, et al. (1992) supra; Mariani, et al. (2000) J. Thorac. Cardiovasc. Surg. 120:660-667; Edwards, et al. (1992) Ann. NY Acad. Sci. 1019:85-95). Optimal therapeutic interventions to antagonize aging should prevent cell death and accumulation of senescent myocytes (Sussman, et al. (2004) supra).

Although the mechanism inducing extension of the maximum life-span may slow down aging of the whole organism, whether or not a defined longevity factor really prevents the aging process of organs and cells has not been clearly demonstrated. It has been shown that heart-specific expression of a molecule known to induce life-span extension (dPTEN and dFOX0) prevents age-dependent decline in the heart rate of Drosophila (Wessells, et al. (2004) Nat. Genet. 36:1275-1281). Moreover, transfection of myocytes with a Sir2α overexpression plasmid and treatment with resveratrol, a Sir2 activator, prevents myocyte cell death in vitro (Pillai, et al. (2005) J. Biol. Chem. 280:43121-43130). This data suggests that regulators of life-span extension can function autonomously to prevent aging of the organ. However, whether or not such a mechanism exists in the mammalian heart has not been demonstrated.

Yeast Sir2 is an evolutionarily conserved molecule which mediates life-span extension in yeast and Drosophila (Rogina and Helfand (2004) supra). Sir2 is an NAD⁺-dependent HDAC, a member of the class III HDAC family, and functions in a wide array of cellular processes, including gene silencing, rDNA recombination, life-span extension, and DNA damage repair (see, Wood, et al. (2004) supra; Smith (2002) Trends Cell Biol. 12:404-406; Blander and Guarente (2004) Annu. Rev. Biochem. 73:417-435) Sir2 deacetylates not only histones but also non-histone substrates, thereby mediating divergent cellular functions. For example, the mammalian homologue of Sir2 deacetylates p53 and the FOX0 family of Forkhead transcription factors, thereby inhibiting apoptotic cell death in mice and humans (Daitoku, et al. (2004) supra; Smith (2002) supra; Luo, et al. (2001) Cell 107:137-148; Vaziri, et al. (2001) Cell 107:149-159; Brunet, et al. (2004) Science 303:2011-2015; Motta, et al. (2004) Cell 116:551-563). Sir2 forms a complex with the acetyltransferase PCAF and MyoD, and retards skeletal muscle differentiation (Fulco, et al. (2003) Mol. Cell 12:51-62). Mice deficient in Sir2α, a murine ortholog of Sir2, exhibit developmental abnormality in the heart and only infrequently survive postnatally (Cheng, et al. (2003) Proc. Natl. Acad. Sci. USA 100:10794-10799), suggesting that Sir2α may possess important functions in the heart. Sirt1* is upregulated by calorie restriction, which in turn regulates fat metabolism by inhibiting fat cell differentiation and fat accumulation through suppression PPAR-γ (Picard, et al. (2004) Nature 429:771-776).

### SUMMARY OF THE INVENTION

The present invention relates to a method for producing a transgenic animal with cardiac-specific overexpression of Sir2α. The method involves introducing a polynucleotide encoding Sir2α into an animal cell, wherein said polynucleotide is operably linked to a cardiac-specific promoter and allowing said cell to develop into a transgenic animal which exhibits cardiac-specific overexpression of the Sir2α polypeptide.

A method for preventing stress- or age-induced cardiac cell death by administering an effective amount of a nucleic acid molecule encoding Sir2α protein to a cardiac cell is also provided as well as a method for identifying a cardioprotective agent by screening for agents that increase the expression or activity of Sir2α.

### BRIEF DESCRIPTION OF THE DRAWINGS

FIG. 1 shows the effects of myocyte-specific overexpression of Sir2α. Myocytes were transduced with an adenoviral vector encoding LacZ (Ad-LacZ) or Sir2α (Ad-Sir2α) with or without serum for 48 hours followed by Cell Death ELISA (FIG. 1A). Alternatively, myocytes were transduced Ad-LacZ or Ad-Sir2α with or without H₂O₂ (100 μM) for 48 hours followed by Cell Death ELISA (FIG. 1B). FIG. 1C shows the relative cell death of cells treated with 10 mM each of pyruvate or lactate for 48 hours, wherein cell death was determined by Cell Death ELISA. Mean OD value of control was designated as 1. Each column represents mean from 3 different culture preparations.

FIG. 2 shows the anti-apoptotic effects of Sir2α overexpression. Three, four-month old male mice transgenic for Sir2α (Tg-Sir2α), and their non-transgenic littermates (NTg), were analyzed. TUNEL-positive myocytes/nuclei were determined at baseline and in response to pressure overload for 10 days (FIG. 2A). There was no significant difference in the pressure gradient, LV weight/body weight and LVEF (%) between NTg and Tg-Sir2α. FIG. 2B shows that the expression of Bcl2 and Bcl-XL was elevated as determined by immunoblot analysis. Experiments were conducted using high expression line 40.

FIG. 3 shows that aging markers are downregulated in Tg-Sir2α mice. Expression levels of senescence markers, p15 (FIG. 3A) and p19 (FIG. 3B), in Tg-Sir2α and NTg hearts are shown. n=4-6. Values are presented as means±SEM.

FIG. 4 shows the expression of cell protective molecules and regulator of cellular senescence/aging in Tg-Sir2α. In these experiments, 3-4 month old Tg-Sir2α mice (line 40) or non-transgenic littermate controls were used. The relative expression of Hsp40 is shown FIG. 4A as determined by densitometric analyses. Similar results were obtained for Hsp90. FIGS. 4B-4C respectively show the relative expression of Tert (telomere reverse transcriptase) and Trf2 (TTAGGG repeat-binding factor 2) as determined by densitometric analyses. FIG. 4D shows the relative expression of Wrn (Werner syndrome protein) as determined by densitometric analyses. In all experiments, α-actin expression was used as a loading control.

FIG. 5 shows apoptotic effects of overexpression of constitutively active (CA) FOXO1 (FOXO1-ADA) in cultured cardiac myocytes. As determined by TUNEL staining, FOXO1-ADA alone induced apoptosis, while co-expression with Sir2α prevented apoptosis.

FIG. 6 shows that Sir2α upregulates the expression of catalase in cultured cardiac myocytes. FIG. 6A shows that when Sir2α is overexpressed by adenovirus transduction (10 MOI), expression of catalase was increased. Similarly, constitutively active FOXO 1a (FOXO-CA) also increases catalase expression; however, Sir2α and FOXO-CA failed to exhibit additive effects. Ad-LacZ was used as control. FIG. 6B shows that Sir2α stimulates promoter activity of catalase. Myocytes were transfected with catalase-Luc reporter gene, SV40 β-gal, together with either control pcDNA3.1 or Sir2α. FIG. 6C shows that catalase expression is upregulated in Tg-Sir2α (line 40, 3 month old) compared with non-transgenic littermates.

FIG. 7 shows echocardiographically determined LVEF in Tg-Sirt1 and NTg hearts at 17 months. n=9.

FIG. 8 shows that Tg-Sirt2α mice are more resistant to oxidative stress. Tg-Sirt2α (n=6) and NTg (n=4) mice from line 40 were treated with ip injection of 10 mg/kg of paraquat (PQ) for 2 weeks. LV ejection fraction (EF, FIG. 8A) and % fractional shortening (% FS, FIG. 8B) of Tg-Sirt2α and NTg mice after PQ treatment is shown. FIG. 8C shows the percent apoptosis in Tg-Sirt2α and NTg hearts as determined by the number of TUNEL positive myocytes divided by the number of DAPI positive nucleus in six separate fields. n=4-5 in each group. FIG. 8D shows malondialdehyde (MDA) content, an indicator of lipid peroxidation, in untreated and PQ treated hearts (n=5). FIG. 8E shows the relative protein expression of catalase in Tg-Sirt2α and NTg hearts after PQ treatment (n=5), whereas FIG. 8F shows the role of FOXO1a in mediating Sirt2α-induced upregulation of catalase in cultured cardiac myocytes. Immunoblot analyses of catalase were conducted, using cardiac myocytes transduced with adenovirus vectors harboring constitutively actively FOXO1a (F-CA), dominant negative FOXO1a (F-DN), or Sirt2α. n=3-4. Values are presented as means±SE.

### DETAILED DESCRIPTION OF THE INVENTION

Yeast Sir2, an NAD⁺-dependent HDAC and a member of the HDAC class III family, functions in a wide array of cellular processes, including gene silencing, longevity, and DNA damage repair. It has now been found that Sir2α, a mouse homologue, plays an essential role in mediating cell survival in cardiac myocytes in vitro (Alcendor, et al. (2004) Circ Res. 95:971-980). More particularly, it has now been found that expression of Sir2 (silent information regulator 2) in cultured cardiac myocytes inhibits death of cardiac myocytes in response to serum starvation. Inhibition of endogenous Sir2 causes apoptosis of cardiac myocytes through p53-dependent mechanisms, indicating that endogenous Sir2 is an essential mediator of cell survival in cardiac myocytes.

It has also been found that expression of Sir2 is increased in the heart from animals with congestive heart failure. Overexpression of Sir2 in transgenic mouse hearts reduces apoptosis at basal conditions and in response to pressure overload. Overexpression of Sir2 in transgenic mouse hearts increases expression of cell survival molecules, including Bcl-2, Bcl-xL, Dnajb and telomerase reverse transcriptase. Overexpression of Sir2 in transgenic mouse hearts reduces expression of the aging senescence marker, pl9ARF. Since aging increases the risk of diseases and reduces the function of organs, the identification of mechanisms controlling organ-specific aging was determined.

To demonstrate that Sir2α is expressed in cardiac cells, nuclear and cytoplasmic fractions prepared from neonatal rat cardiac myocytes were subjected to immunoblotting with anti-murine Sir2α antibody. A 110 kD protein was detected predominantly in the nuclear fraction. To examine the function of Sir2α in cardiac myocytes, nicotinamide (NAM) and sirtinol, inhibitors of Sir2 (Bitterman, et al. (2002) J. Biol. Chem. 277:45099-45107; Grozinger, et al. (2001) J. Biol. Chem. 276:38837-38843) were employed. Myocytes were also treated with trichostatin A (TSA), an inhibitor of class I and II HDACs (Johnstone (2002) Nat. Rev. Drug Discov. 1:287-299). Treatment of cardiac myocytes with NAM or TSA increased acetylation of Histones 3 and 4 (Imai, et al. (2000) supra), indicating that both NAM and TSA inhibit HDACs. Treatment with NAM for 48 hours caused dose-dependent increases in cell shrinkage and detachment, consistent with apoptosis. Similar findings were also obtained with sirtinol. By contrast, TSA modestly increased cell size, although it also caused death in some myocytes at high doses.

Subsequently, it was determined whether cardiac myocyte death by Sir2α inhibition was due to apoptosis. Both NAM and sirtinol significantly increased mono- and oligo-nucleosomes in the cytoplasm, a sensitive marker of apoptosis (Yamamoto, et al. (2001) J. Mol. Cell Cardiol. 33:1829-1848). Although TSA also significantly increased nuclear fragmentation, it was not as potent as NAM. Similar results were obtained using TUNEL staining. To examine whether activation of caspase-3 was involved in myocyte death induced by NAM and TSA, the effect of DEVD-CHO, a cell permeable inhibitor of caspase-3, upon myocyte death was examined. DEVD-CHO prevented increases in TUNEL positive myocytes caused by NAM. In contrast, DEVD-CHO did not affect a modest increase in TUNEL-positive myocytes due to TSA. NAM and sirtinol induced cleavage of caspase-3, while TSA did not. These results indicate that cardiac myocyte death induced by inhibition of Sir2α was caspase-dependent (apoptosis), while a modest increase in cell death by TSA was caspase-independent.

To further confirm that inhibition of endogenous Sir2α stimulates cardiac myocyte apoptosis, the effects of a dominant-negative Sir2α were examined. Transduction with Ad-dominant-negative Sir2α enhanced cell death of myocytes, as determined by TUNEL staining, in serum-free cultures and in response to H₂O₂ (0.1 mM) compared with Ad-LacZ. The level of cleaved caspase-3 was higher in dominant-negative Sir2α-transduced myocytes than in LacZ-transduced cells. These results confirm that inhibition of the HDAC activity of Sir2α enhances cardiac myocyte apoptosis.

The affects of cardiac-specific inhibition of endogenous Sir2α on p53, a positive mediator of apoptosis, were also examined. Treatment of myocytes with NAM dose-dependently increased the activity of p53-Luc. The effect of NAM was specific to the p53 DNA binding sequence because the control reporter gene did not respond to NAM. By contrast, TSA failed to activate p53-Luc. To examine whether the status of acetylation in p53 is affected by NAM or TSA in cardiac myocytes, nuclear fractions were subjected to immunoblot analysis with anti-acetylated p53 antibodies. Acetylation of p53 was increased at both K373/382 (1.8-fold) and K320 (2.8-fold) in myocytes treated with NAM but not with TSA, indicating that endogenous Sir2α plays an important role in inhibiting acetylation of p53 in cardiac myocytes. Neither NAM nor TSA significantly changed the total amount of p53. Taken together, these results indicate that inhibition of Sir2α increases acetylation of p53 and stimulates its transcriptional activity.

To examine whether cardiac myocyte apoptosis due to inhibition of Sir2α is mediated by p53, cardiac myocytes were transduced with adenovirus harboring temperature-sensitive dominant-negative p53 and cultured at 39° C. Dominant-negative p53 slightly reduced cell shrinkage and nuclear fragmentation due to serum starvation alone, but this did not reach statistical significance. By contrast, dominant-negative p53 significantly reduced cell shrinkage and death caused by NAM. Ad-LacZ failed to rescue NAM-induced cell shrinkage/death. The temperature-sensitive dominant-negative p53 construct works as a dominant-negative only at high temperatures (Regula and Kirshenbaum (2001) J. Mol. Cell. Cardiol. 33:1435-1445). Consistent with this, the cell death inhibitory effect of dominant-negative p53 against NAM treatment was not observed at 32° C. These results indicate that p53 plays an essential role in mediating myocyte apoptosis induced by NAM.

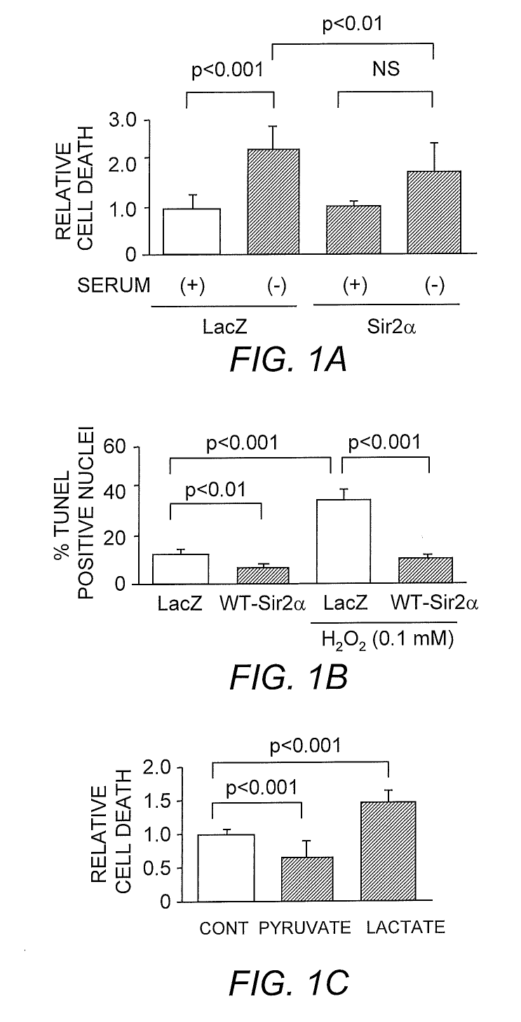

Cardiac-specific overexpression of Sir2α was subsequently examined. Wild-type cardiac myocytes exhibited shrinkage and cell death under serum-free conditions. After 7 days, most myocytes were dead in a low-density culture condition. In contrast, overexpression of Sir2α (10 MOI, 4.7-fold) under these same conditions significantly inhibited shrinkage and death of cardiac myocytes. Transduction of Ad-LacZ did not prevent cell shrinkage or death. Quantitative analyses showed that myocytes cultured in serum-free conditions for 48 hours had significantly increased cytoplasmic accumulation of mono- and oligo-nucleosomes compared with those in serum-containing medium. Overexpression of Sir2α inhibited increases in nuclear fragmentation induced by serum starvation compared with that of Ad-LacZ (FIG. 1A). Overexpression of Sir2α also inhibited cardiac myocyte apoptosis in response to H₂0₂ stimulation as determined by cell death ELISA assays (FIG. 1B). It has been shown that pyruvate increases, while lactate decreases, the cellular NAD⁺/NADH ratio, thereby positively or negatively regulating the activity of endogenous Sir2α (Fulco, et al. (2003) Mol. Cell 12:51-62). Consistent with this, pyruvate significantly inhibited cytoplasmic accumulation of mono- and oligo-nucleosomes induced by serum starvation, while lactate stimulated it (FIG. 1C). These results indicate that the increased activity of either exogenous or endogenous Sir2α protects cardiac myocytes from apoptosis caused by serum starvation.

The affect of pathologic stimuli on protein expression of Sir2α in the heart was also analyzed. Immunoblot analyses were conducted using left ventricular homogenates obtained from dogs with sham operation, compensated LVH, and LVH plus heart failure (Hittinger, et al. (1989) Circ. Res. 65:971-980). Expression of a Sir2α-like protein was significantly elevated in the failing hearts, thereby indicated that this longevity factor is upregulated in response to stresses.

To further analyze the effects of Sir2α in vivo, transgenic mice with cardiac-specific overexpression of Sir2α (Tg-Sir2α) were generated, using the α MHC promoter. Six transgenic lines with different expression levels were generated. Initial characterization of line 40 indicated that the level of Sir2α in the heart was 10-fold higher than in non-transgenic (NTg) littermates. The level of acetylated histone H3 was lower in Tg-Sir2α than in NTg, indicating that the transgenic Sir2α was active in Tg-Sir2α mice. Since extremely high levels of overexpression could cause some undesirable physiological effects, mild overexpression lines were also developed. One of the lines (line 39) exhibited 2.5-fold overexpression of Sir2α. In this analysis, the mice were followed for up to 12 months. Characterization of these mice was conducted using lines 39 and 40. The mice showed no apparent base-line cardiac phenotype: left ventricular weight/tibial length and lung weight/tibial length were normal. Echocardiographically determined cardiac dimensions and left ventricular ejection fraction were normal as well.

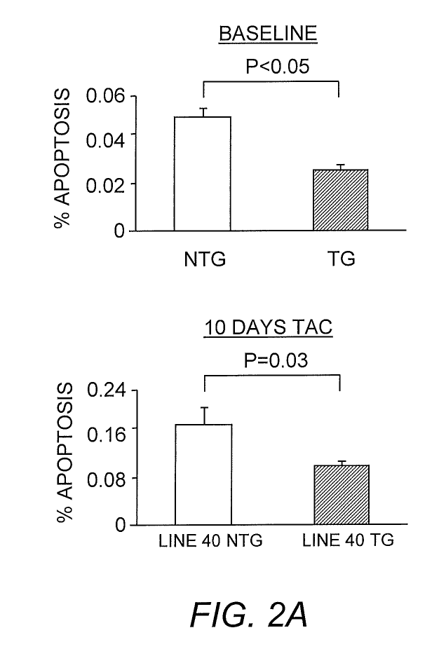

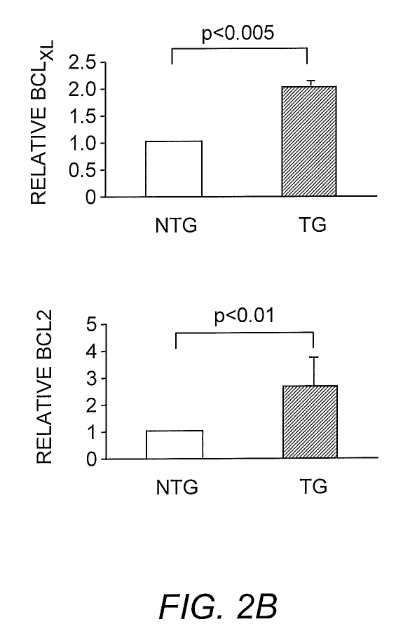

TUNEL-positive cells/total nuclei was significantly less in Tg-Sir2α compared to NTg littermates at baseline (FIG. 2A). Unexpectedly, expression of anti-apoptotic molecules, including Bcl-2 and Bcl-xL, was elevated in Tg-Sir2α compared with NTg littermates (FIG. 2B). To examine the effect of stress upon the extent of apoptosis, thoracic aortic banding was applied for 10 days according to established methods (Sadoshima, et al. (2002) J. Clin. Invest. 110:271-279; Yamamoto, et al. (2003) J. Clin. Invest. 112:1395-1406). After 10 days, there was no significant difference in LV weight/tibial length between Tg-Sir2α and NTg. Although aortic banding increased TUNEL-positive cells in both NTg and Tg-Sir2α, Tg-Sir2α exhibited fewer TUNEL-positive cells than NTg (FIG. 2A).

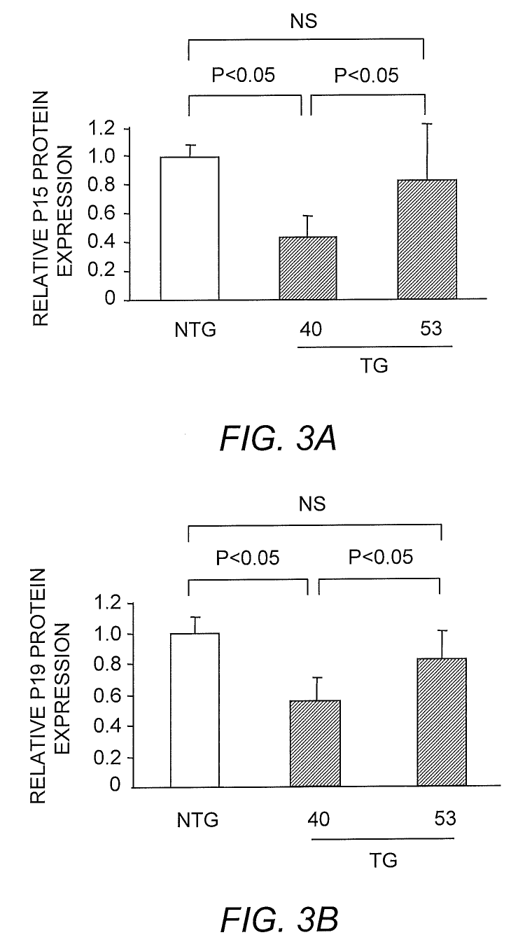

Expression of the INK4/ARF family proteins, inducers of cellular senescence, markedly increases with advancing age in many organs in rodents, including the heart. Thus, it was determined whether expression of Sir2α modulates the expression of inducers of cellular senescence. The hearts Tg-Sir2α and NTg male littermates were harvested and expression of p19^{ARF} and p15^{INK4b} was examined. Unexpectedly, Expression of p15^{INK4b} and p19^{ARF} was significantly lower in Tg-Sirt1 line 40, but not in Tg-Sirt1 line 53, compared with NTg controls (FIGS. 3A and 3B). This result is consistent with the ability of Sir2α to inhibit the progression of aging in the mouse heart in vivo.

DNA microarray analysis was also conducted to examine whether mechanisms mediating anti-aging or stress-resistant effects of Sir2α were affected in Tg-Sir2α at baseline. Three Tg-Sir2α (high expression line 40) and four non-transgenic littermates (245 days old) were analyzed. There was no prominent baseline phenotype and cardiac function was normal. Genes with more than a 1.4-fold change were screened. A subset of genes was consistently either up- or down-regulated in Tg-Sir2α (Table 1). For example, cell protective molecules (Hsp40, thrombospondin 3, angiopoietin-like 1) and regulators of aging (telomerase reverse transcriptase (Tert), Werner syndrome proteins (Wm)) and protein kinases (JAK3 homologue) were upregulated, while proapoptotic molecules (Ca²⁺ channel, Granzyme D) were down-regulated.

TABLE 1

| Up-Regulated in Tg-Sir2α | Down-Regulated in Tg-Sir2α |
|---|---|
| Transcription Factors | Pro-apoptotic Factors |
| Foxl1, Foxp2, Elk4 | Ca²⁺ channel, CHK2, NO |
| Known Regulators of Aging | synthase 3, GzmD, PAK |
| Tert, Wm | Regulator of Aging |
| Cell Protective Molecules | Shc homologue |
| DnaJ (Hsp40), Mas, G6pd2, | GPCRs |
| Thbs2, Angpt11, IL1RA, p27 | GABA-A, Adra2b, Adenosine |
| Tumor Suppressor Genes | A2a |
| Lats2, p68 | |
| Protein Kinases | |
| Btk, Rps6Kb2, JAK3 | |
| homologue, Src homologue | |

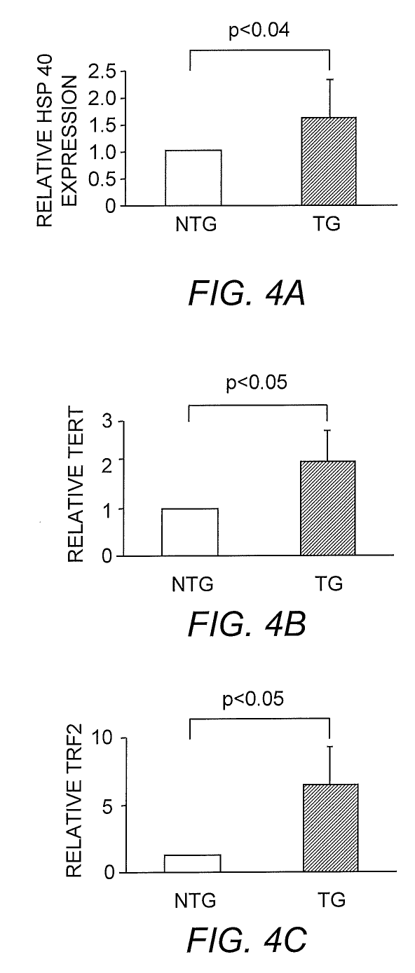

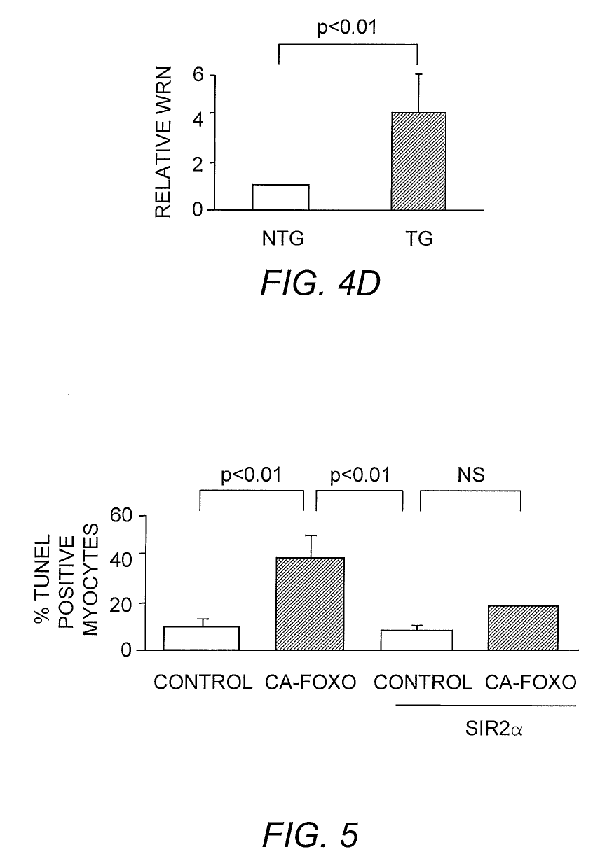

Using RT-PCR, the expression of small heat shock proteins (Hsp40 and Hsp90) (FIG. 4A), Tert (FIG. 4B) and telomere capping protein (Trf2)(FIG. 4C), Wrn (FIG. 4D) was confirmed in Tg-Sir2α. Upregulation of Hsps may protect the heart from stresses while upregulation of Tert and Trf2 would stimulate the function of telomere. Mutations in Wrn could cause progeric syndrome due to its defect in the helicase activity.

The involvement of FOX0 family transcription factors in mediating the effect of Sir2α in neonatal rat cardiac myocytes was also examined. Adenovirus vectors harboring wild-type FOXOla, constitutively active FOXOla (FOXO1-ADA, hereafter termed CA-FOXO1) or FOXOl (Δ256) (dominant negative (DN)-FOXOl) were transduced into neonatal rat cardiac myocytes. While cardiac myocytes express endogenous FOXOl, exogenous FOXOl was successfully overexpressed. Immunofluorescent staining of FOXOl indicated that CA-FOX01 was localized to the nucleus (Nakae, et al. (2001) J. Clin. Invest. 108:1359-1367) and CA-FOX01 expression increased expression of Sir2α in cardiac myocytes (Nemoto, et al. (2004) Science 306:2105-2108). Although expression of CA-FOX01 caused shrinkage and death of myocytes, consistent with apoptosis, co-expression of Sir2α prevented myocardial cell death (FIG. 5). Furthermore, wild-type Sir2, but not DN-Sir2α, increased protein expression of FOXOla. These results indicated that Sir2α and FOXO affect the function of one another in cardiac myocytes.

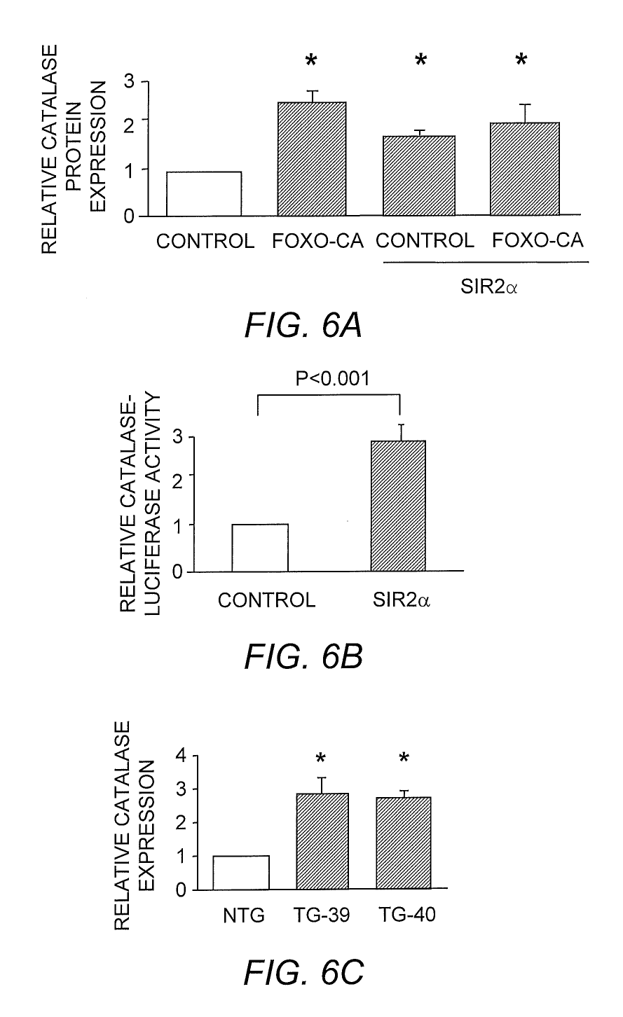

To examine the mechanism mediating the beneficial effects of Sir2α, the effect of Sir2α upon expression of catalase was determined. Adenovirus-mediated expression of Sir2α, but not LacZ, induced expression of catalase in cardiac myocytes (FIG. 6A). Co-transfection experiments indicate that Sir2α increases the activity of the catalase-Luc reporter gene (Luo and Rando (2003) Biochem. Biophys. Res. Commun. 303:609-618), indicating that Sir2α can stimulate transcription through the catalase promoter (FIG. 6B). To examine the role of FOXO, CA-FOXO1 was also expressed. CA-FOXOl also increased expression of catalase in cardiac myocytes (FIG. 6A). Sir2α and CA-FOXOl did not show additive effects, indicating that they act on the same signaling pathway (FIG. 6A). Significant upregulation of catalase was also observed in Tg-Sir2α (FIG. 6C). Together with the fact that Sir2α and FOXOla stimulate expression of one another, these results indicate that Sir2α and FOXOl act in to concert regulate catalase expression in cardiac myocytes.

To control the timing of Sir2α or dominant-negative Sir2α overexpression in post-natal hearts, tetracycline-regulatable (Tet-off) transgenic mice are generated using a commercially available vector system (Sanbe, et al. (2003) Circ. Res. 92:609-616). Transgenic constructs for wild-type and dominant-negative Sir2α were prepared and 17 wild-type Sir2α responder lines were generated. Several lines were crossed with cardiac-specific tTA mice (Tg-α-MHC-tTA) (Sanbe, et al. (2003) supra) and induction of Sir2α in the heart and suppression by doxycycline (Dox) treatment was confirmed in bigenic mice from at least one line. The bigenic mice expressing Sir2α showed no significant basal cardiac phenotype, consistent with the findings from non-conditional Tg-Sir2α. To avoid expression of the transgene during fetal developmental stages, rodent chow containing 2 mg/g Dox is applied to pregnant mothers.

It is widely accepted that oxidative stress promotes aging (Finkel and Holbrook (2000) Nature 408:239-247). Transgenic mice, which systemically overexpress thioredoxin (Trx1), live longer than control littermates (Mitsui, et al. (2002) supra; Murata, et al. (2002) supra). Thus, transgenic mice with cardiac-specific overexpression of Trxl (Tg-Trxl) were analyzed (Yamamoto, et al. (2003) supra). Although Tg-Trxl did not show significant baseline phenotype, they showed a significantly smaller increase in oxidative stress and hypertrophy in response to pressure overload (Yamamoto, et al. (2003) supra). To examine age-dependent changes in the mouse heart, echo-cardiographic analyses were conducted using 12-15 month-old male mice. Unexpectedly, LVEF and LV % FS in Tg-Trx1 were significantly better than in NTg littermates. Histological analyses indicated that Tg-Trx1 exhibited significantly less apoptosis than non-transgenic littermates.

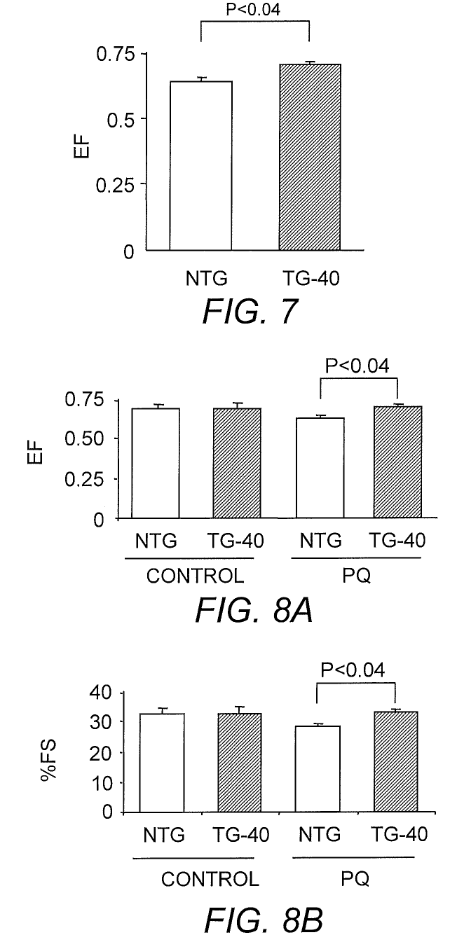

To examine the effect of Sirt1 (i.e., Sir2α) overexpression upon progression of aging in the heart, a histopathological examination of older line 40 mice, namely an age group of 15-19 months (mean age 17 months) was carried out. The extent of both cardiac myocyte apoptosis and myocardial fibrosis in NTg was significantly greater in the 17-month old than in the 6-month old group. However, the parameters in the 17-month old group were significantly milder in Tg-Sirt1 line 40 than in NTg. Furthermore, LVEF in Tg-Sirt1 (line 40) at 17 months old was significantly greater than that in old NTg (FIG. 7). These results indicate that aging of the heart is retarded in Tg-Sirt1 line 40.

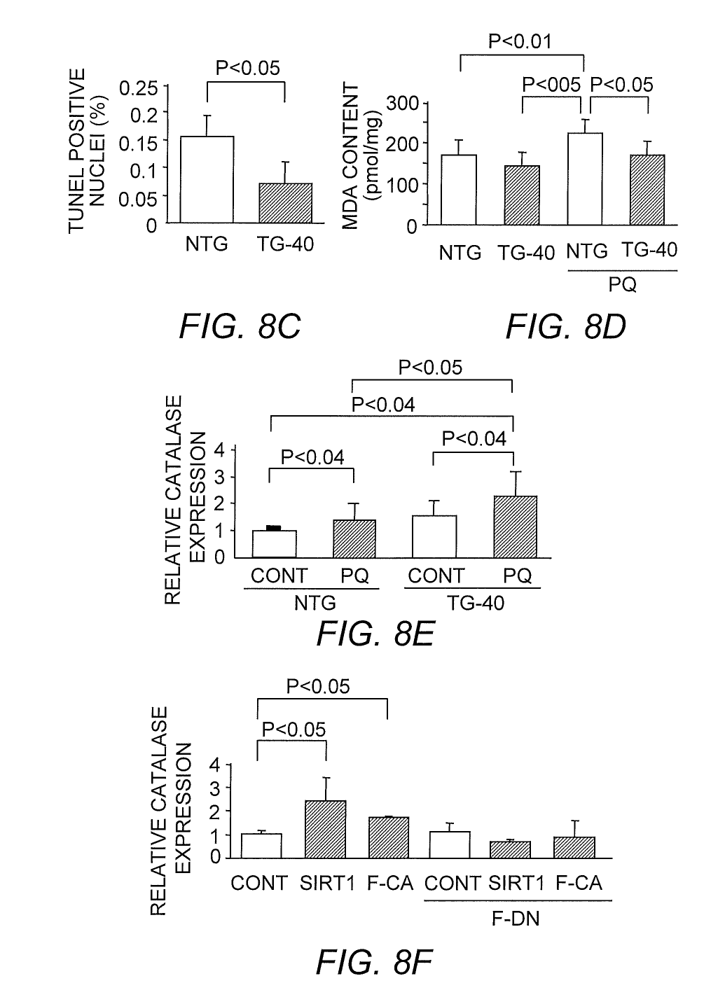

Since Tg-Sirt1 line 40 exhibited reduced levels of baseline apoptosis and aging markers, it was determined whether modest overexpression of Sirt1 was protective against stress. To this end, mice were treated for 14 days with paraquat, an inducer of oxidative stress. Paraquat treatment significantly reduced LV contraction in NTg control but not in Tg-Sirt1 line 40 (FIGS. 8A and 8B). Furthermore, the extent of LV cardiac myocyte apoptosis after paraquat treatment was significantly greater in NTg compared to that in Tg-Sirt1 (FIG. 8C). Paraquat treatment significantly increased the extent of oxidative stress in NTg mouse hearts as determined by 8-hydroxy-2′-deoxyguanosine (8-OHdG) staining as well as cellular malondialdehyde (MDA) content, established markers of oxidative stress. The paraquat-induced increases in oxidative stress were abolished in Tg-Sirt1 line 40 (FIG. 8D). These results indicate that increased oxidative stress and subsequent myocardial damage induced by paraquat was significantly attenuated by Sirt1 overexpression. Paraquat-induced increases in expression of catalase, an antioxidant, were greater in Tg-Sirt1 than in NTg hearts (FIG. 8E). Overexpression of Sirt1 or constitutively active FOXO1a in cultured cardiac myocytes stimulated expression of catalase, whereas upregulation of catalase was inhibited in the presence of dominant negative FOXO1a (FIG. 8F). These results indicate that FOXO1a plays an important role in mediating Sirt1-induced upregulation of catalase, which may in part mediate suppression of myocardial damage due to oxidative stress in Tg-Sirt1 line 40.

A 45 minute ischemia and 24 hour reperfusion was also applied to the mouse heart in vivo. After reperfusion the extent of apoptosis was significantly smaller in Tg-Sirt1 mice compared to NTg.

An adenovirus harboring shRNA targeting GSK-3β and Beclin 1 was designed and cloned into pSILENCER 1.0 (Ambion) and an adenovirus vector harboring shRNA targeting GSK-3β and Beclin 1 was generated. Myocytes were transduced with the adenovirus (10-30 MOI) and 4-6 days after transduction, specific and significant reduction in GSK-3β and Beclin 1 was observed.

Another important component of these studies was the establishment of adult rat cardiac myocyte cultures. This was achieved using the method of cardiac myocyte dissociation originally described by Zhou et al (Zhou, et al. (2000) Am. J. Physiol. Heart Circ. Physiol. 279:H429-436) with modifications (Mitcheson, et al. (1998) Cardiovasc. Res. 39:280-300). At 12 hours, cardiac myocytes were transduced with adenovirus harboring green fluorescent protein (GFP), which was shown to be expressed at 72 hours in the adult rat cardiac myocytes.

Having demonstrated a role for Sir2α, also referred to herein as Sirt1, in organ-specific longevity, the present invention relates to the exogenous expression or endogenous stimulation of the expression or activity this longevity factor in the heart thereby providing organ-specific protection against responses to stress and aging-related pathologic changes. This represents a new method of cardioprotection, which can be applied to the treatment of heart diseases, such as ischemic heart disease and congestive heart failure. Moreover, transgenic animals with cardiac-specific (constitutive or conditional) overexpression of Sir2α provide a means for analyzing the aging heart and various means for retarding or slowing down the aging process.

Accordingly, the present invention embraces a method for producing a transgenic animal with cardiac-specific overexpression of Sir2α. As used herein, the term transgenic refers to the introduction of an exogenous nucleic acid molecule into a cell so that the nucleic acid molecule is incorporated into the genome of the cell. The cell may be capable of giving rise to a transgenic animal which contains the transgenic nucleic acid molecule. Generally, the transgenic nucleic acid molecule for administration into a particular cell can be constructed using a transgenic vector. A preferred nucleic acid molecule is a polynucleotide that encodes for full-length Sir2α or an analog thereof. Nucleic acid molecules encoding full-length Sir2α are well-known in the art. For example, nucleic acid and protein sequences for murine Sir2α are disclosed in GENBANK Accession No. AY377984, whereas nucleic acid and protein sequences for human Sir2α are disclosed in GENBANK Accession No. AF235040.

A recombinant nucleic acid molecule or sequence simply means the nucleic acid molecule or sequence has been manipulated in any one of a number of recombinant DNA techniques known in the art. Likewise, there are many known processes for generating transgenic animals. These processes are essentially the same regardless of the species involved. While the following disclosure describes transgenic mice, the same techniques can be used to produce non-mouse transgenic animals, and their creation and use is encompassed within the scope of this invention.

One process begins with transgenic nucleic acid molecule operably linked to a promoter. The transgenic nucleic acid molecule-promoter complex is introduced into the pronuclei of a fertilized egg of a non-human animal. The egg is then implanted into a pseudopregnant non-human animal and allowed to develop into a transgenic animal. Fertilized eggs from a variety of animals used in the above described method can be produced using techniques well-known to those of ordinary skill in the art. For example, the use of bovine oocytes to produce embryos is described in Dominko, et al. (1999) Biol. Reprod. 60(6):1496-502. Alternatively, fertilized eggs from mice, rabbits, and sheep, as well as other animals can be obtained (Mullins, et al. (1996) J. Clin. Invest. 98:S37 S40). Accordingly, the invention is applicable to animals other than the specifically exemplified mice.

A second method for producing transgenic animals involves the modification of embryonic stem (ES) cells. This second method involves introducing transfected cells into embryos at a stage at which they are capable of integrating into the embryo, for example, at the blastocyte stage. The embryo with transfected cells is then replanted into a surrogate mother, resulting in chimeric offspring possessing the transgenic nucleic acid molecule. Embryonic stem cells are available from a number of sources. These include mice, rats, cows, pigs, sheep, and other animals (Joyner, ed. (1993) Gene Targeting: A Practical Approach, New York: Oxford University Press). See also, Hogan, et al., eds. (1994) Manipulating the Mouse Embryo: A Laboratory Manual, Cold Spring Harbor Laboratory Press, which describes manipulating the mouse embryo. Methods used in successful research with rat ES cells are described by Iannaccone, et al. ((1994) Dev. Biol. 163(1):288-92), whereas the use of rabbit ES cells is described by Schoonjans, et al. ((1996) Mol. Reprod. Dev. 45(4):439-43). In addition, Couly and Le Douarin ((1990) Development 108:543-555) describe methods for isolating and manipulating chicken and quail embryos. Kimmel and Warga ((1987) Nature 327:234-237) describe isolation and manipulation of zebrafish embryos, whereas Ware, et al. ((1988) Society for the Study of Reproduction 38:241) also discusses an embryonic stem cell culture condition amenable for many species like mouse, cattle, pig, and sheep. Specific references for the generation of transgenic pigs from embryonic stem cells include Notarianni, et al. ((1997) Int. J. Dev. Biol. 41(3) :537-40) and Gutierrez-Adan, et al. ((1997) Biol. Reprod. 57(5) :1089-95). Specific references for the generation of transgenic cows from embryonic stem cells include Cibelli, et al. ((1998) Nat. Biotechnol. 16(7):642-6) and Kubota, et al. ((2000) Proc. Natl. Acad. Sci. USA 97(3):990-5). Preparing primate embryonic stem cells can be facilitated by referring to Thompson, et al. ((1995) Proc. Natl. Acad. Sci. USA 92(17):7844-8).

Various methods are known in the art for introducing nucleic acid molecules into animal cells, for example, ES cells. Transgenic nucleic acid molecules can be microinjected into the appropriate cells. Also, viral vectors can be used to introduce the DNA into appropriate cells and the genome of those cells (see, for example, Tsukui, et al. (1996) Nature Biotechnology 14:982-985). Alternatively, cells can be manipulated in vitro through transfection and electroporation methods (see, for example, Ausubel, et al., eds. (1989) Current Protocols in Molecular Biology, Greene Publishing Associates and Wiley-Interscience, John Wiley & Sons, Boston, Mass.; Hogan, et al., eds. (1994) Manipulating the Mouse Embryo: A Laboratory Manual, Cold Spring Harbor Laboratory Press). While transgenic nucleic acid molecules can be incorporated into a cell genome through random integration, homologous recombination is also possible.

The design of transgenic vectors involves operably linking the transgenic nucleic acid molecule to an appropriate cardiac-specific promoter sequence and cloning the fusion molecule into an appropriate vector. A cardiac-specific promoter is intended to mean that expression is limited to cardiac tissue, with minimal or no significant expression in any other tissue types. A nucleic acid molecule is said to operably linked to a promoter when the promoter regulates the timing and/or level of expression of the mRNA transcribed from the nucleic acid molecule. Examples of promoters of use in accordance with the present invention include, but are not limited to, alpha-myosin heavy chain promoter which gives cardiac myocyte-specific expression (see, e.g., Kang, et al. (2005) J. Nucl. Med. 46(3):479-83). As disclosed herein, cardiac-specific overexpression of the Sir2α can be constitutive or conditional, i.e., regulated by the presence of small molecules such as tetracycline.

Increasing or enhancing cardiac expression of Sir2α in a transgenic animal model is useful as a research tool for studying cardiac responses to stress and aging. As such, overexpression has been shown to decrease apoptosis and increase cardiac cell longevity. Therefore, the present invention also embraces increasing the expression or activity of Sir2α in humans to prevent stress- or age-related cardiac cell death thereby facilitating the treatment of cardiac diseases or conditions which are associated with aging (e.g., congestive heart failure).

Therefore, the present invention is also a method for preventing stress- or age-induced cardiac cell death by administering an effective amount of a nucleic acid molecule encoding Sir2α protein to a cardiac cell. Stress-induced or age-induced cell death includes that initiated by such agents as free radicals, toxins and the like. As disclosed herein, nucleic acid molecules encoding a Sir2α protein are well-known in the art and are provided in GENBANK Accession No. AF235040.

In general, administration of an effective amount of a nucleic acid molecule encoding Sir2α protein is achieved cloning the Sir2α nucleic acid molecule into an expression vector suitable for in vivo or ex vivo therapeutic expression (i.e., gene therapy). Such a gene transfer vector includes, but is not limited to, a naked plasmid, a viral vector, such as an adenovirus, an adeno-associated virus, a herpes-simplex virus based vector, a lentivirus vector such as those based on the human immunodeficiency virus (HIV), a vaccinia virus vector, a synthetic vector for gene therapy, and the like (see Miller and Rosman (1992) BioTechniques 7:980-990; Anderson, et al. (1998) Nature 392:25-30; Verma and Somia (1997) Nature 389:239-242; Wilson (1996) New Engl. J. Med. 334:1185-1187; Suhr, et al. (1993) Arch. Neurol. 50:1252-1268). For example, a gene transfer vector employed herein can be a retroviral vector. Retroviral vectors contemplated for use herein are gene transfer plasmids that have an expression construct, i.e., a nucleic acid molecule encoding a Sir2α protein operatively linked to an appropriate promoter and terminator sequence, residing between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764).

Suitable retroviral vectors for use herein are described, for example, in U.S. Pat. Nos. 5,399,346 and 5,252,479; and in WO 92/07573, WO 90/06997, WO 89/05345, WO 92/05266 and WO 92/14829. These documents provide a description of methods for efficiently introducing nucleic acids into human cells using such retroviral vectors. Other retroviral vectors include, for example, mouse mammary tumor virus vectors (e.g., Shackleford, et al. (1988) Proc. Natl. Acad. Sci. USA 85:9655-9659), human immunodeficiency virus (e.g., Naldini, et al. (1996) Science 272:165-320), and the like.

Various procedures are also well-known in the art for providing helper cells that produce retroviral vector particles that are essentially free of replicating virus. See, for example, U.S. Pat. No. 4,650,764; Miller (1990) Human Gene Therapy 1:5-14; Markowitz, et al. (1988) J. Virol. 61(4):1120-1124; Watanabe, et al. (1983) Mol. Cell. Biol. 3(12):2241-2249; Danos, et al. (1988) Proc. Natl. Acad. Sci. USA, 85:6460-6464; and Bosselman, et al. (1987) Mol. Cell. Biol. 7(5): 1797-1806, which disclose procedures for producing viral vectors and helper cells that minimize the chances for producing a viral vector that includes a replicating virus.

An exemplary gene transfer vector is a replication-deficient adenovirus carrying a nucleic acid molecule encoding a Sir2α protein to effect increases in cardiac-specific expression in a subject. When used in combination with catheter-mediated infusion, such replication-defective adenovirus vectors have provided prolonged recombinant gene expression in the myocardium (Barr, et al. (1994) Gene Ther. 1(1):51-8; Ding, et al. (2004) Gene Ther. 11(3):260-5). In general, a nucleic acid molecule encoding a Sir2α protein can be transferred into the heart, including cardiac myocytes, in vivo and direct constitutive production of a Sir2α protein.

For ex vivo applications, adult bone marrow cells can be obtained from the subject being treated and grown under suitable culture conditions in a container for a period of time sufficient to promote production by the bone marrow of early attaching cells. The early attaching cells are transfected in culture with a vector as described herein containing a nucleic acid molecule encoding a Sir2α protein and the transfected early attaching cells (and/or medium in which they are cultured after transfection) are then directly administered (e.g., catheter-mediated infusion) to a desired site such as the myocardium in the subject so as to deliver to the site the expressed Sir2α protein.

Depending on the gene transfer vector selected and the mode of administration (i.e., catheter-mediated infusion, i.p. injection, or ex vivo cell delivery), a nucleic acid molecule encoding a Sir2α protein can be operatively linked to a variety of promoters to control initiation of mRNA transcription. Such promoters typically contain at least a minimal promoter in combination with a regulatory element which mediates temporal and/or spatial expression. When constitutive high-level expression is desired and the gene transfer vector is to be infused directly into myocardial tissue, a constitutive promoter such as CMV immediate early, HSV thymidine kinase, early and late SV40 can be selected. When myocardial-specific expression is desired, a myocardial-specific promoter such as an α-myosin heavy chain promoter (Kang, et al. (2005) supra) can be employed.

The results disclosed herein indicate that expression of Sir2α is increased in response to H₂0₂ in cardiac myocytes and during heart failure in the heart. Based upon these lines of evidence, Sir2α is an important molecule mediating cardioprotection; a fact which was demonstrated in the heart in vivo. Therefore, by increasing or enhancing cardiac expression or activity of Sir2α, stress- or age-related cardiac cell death is prevented thereby facilitating the treatment of cardiac diseases or conditions which are associated with aging (e.g., congestive heart failure).

As used herein, cardiac diseases or conditions which are associated with aging are generally diseases or conditions in which damage to heart cells has occurred as a result of the aging process. Such diseases or conditions include, but are not limited to, congestive heart failure, myocarditis, congestive cardiomyopathy, restrictive cardiomyopathy, and cardiac tumors, or other injury or stimuli which damage heart cells.

The terms treat or treatment refer to both therapeutic treatment and prophylactic or preventative measures, wherein the object is to prevent or slow down (lessen) an undesired physiological change or disorder, such as the development of congestive heart failure, cardiomyopathy, etc. For purposes of this invention, administration of an effective amount of an agent disclosed herein results in a beneficial or desired clinical result including, but are not limited to, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Those in need of treatment include those already with the condition or disorder as well as those at risk of having the condition or disorder (e.g., subjects disposed or predisposed to altered cardiac function based on genetics or life-style). In one embodiment, treatment is provided to a subject exhibiting signs or symptoms of heart failure. In another embodiment, treatment is provided to a subject at risk of heart failure as a preventive measure.

In the context of the methods of the present invention, a subject is intended to include any animal classified as a mammal, including humans, domestic and farm animals, as well as zoo, sport, or companion animals, such as dogs, horses, cats, cows, etc.

In addition to the direct cardiac-specific overexpression of Sir2α via expression from exogenous nucleic acid molecules, the present invention also provides for a screening assay to identify cardioprotective agents which increase or enhance the endogenous expression or activity of Sir2α. Such a screening assay involves contacting a cardiac cell (e.g., a cardiac myoctye) with a test agent and determining whether the test agent modulates the expression or activity of Sir2α, wherein an increase in the expression or activity of Sir2α is indicative of a cardioprotective agent.

Agents which increase the expression or activity of Sir2α can be rationally designed from the crystal structure of the protein of interest or identified from a library of test agents. Test agents of a library can be synthetic or natural compounds. A library can comprise either collections of pure agents or collections of agent mixtures. Examples of pure agents include, but are not limited to, peptides, polypeptides, antibodies, oligonucleotides, carbohydrates, fatty acids, steroids, purines, pyrimidines, lipids, synthetic or semi-synthetic chemicals, and purified natural products, derivatives, structural analogs or combinations thereof. Examples of agent mixtures include, but are not limited to, extracts of prokaryotic or eukaryotic cells and tissues, as well as fermentation broths and cell or tissue culture supernatants. In the case of agent mixtures, one may not only identify those crude mixtures that possess the desired activity, but also monitor purification of the active component from the mixture for characterization and development as a therapeutic drug. In particular, the mixture so identified can be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Agents of interest in the present invention are those with functional groups necessary for structural interaction with proteins, particularly hydrogen bonding, and typically include at least an amine, carbonyl, hydroxyl or carboxyl group. The agents often comprise cyclical carbon or heterocyclic structures and/or aromatic or polyaromatic structures substituted with one or more of the above functional groups.

Subsequent to applying the test agent to the host cells encoding Sir2α, the expression or activity of Sir2α is determined. Any method known in the art for determining or measuring Sir2α levels or activity can be used in the present invention. For example, and not by way of limitation, one such method of determining proteins levels is by a quantitative ELISA. Alternatively, fluorescent or enzymatic reporter proteins such as green fluorescent protein or beta-galactosidase can be fused to the Sir2α promoter to monitor Sir2α expression in the presence and absence of a test agent. Moreover, agents which increase the histone deacetylase activity of Sir2α can be identified using well-known histone deacetylase activity assays.

An agent identified in accordance with the instant assay method can be formulated into a pharmaceutically acceptable composition for therapeutic use in accordance with the methods disclosed herein. The agent can be formulated with any suitable pharmaceutically acceptable carrier or excipient, such as buffered saline; a polyol (e.g., glycerol, propylene glycol, liquid polyethylene glycol and the like); carbohydrates such as glucose, mannose, sucrose or dextrans, mannitol; amino acids such as glycine; antioxidants; chelating agents such as EDTA or glutathione; preservatives or suitable mixtures thereof. In addition, a pharmaceutically acceptable carrier can include any solvent, dispersion medium, and the like which may be appropriate for a desired route of administration of the composition. The use of sustained-release delivery systems such as those disclosed by Silvestry, et al. ((1998) Eur. Heart J. 19 Suppl. I:I8-14) and Langtry, et al. ((1997) Drugs 53(5) :867-84), for example, are also contemplated. The use of such carriers for pharmaceutically active substances is known in the art. Suitable carriers and their formulation are described, for example, in Remington: The Science and Practice of Pharmacy, Alfonso R. Gennaro, editor, 20th ed. Lippincott Williams & Wilkins: Philadelphia, Pa., 2000.

Agents that increase the level or activity of Sir2α in the heart can be administered to a subject via various routes. For example, such administration can be carried out by inhalation or insufflation (either through the mouth or the nose), oral, buccal, parenteral, implantation (e.g., subcutaneously or intramuscularly), or directly infused into the myocardium (e.g., via a catheter). A selected agent can be administered continuously or intermittently (e.g., every couple of days, weeks, or months) to achieve the desired effect for an extended period of time.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well-known in the art. Liquid preparations for oral administration can take the form of, for example, solutions, syrups or suspensions, or they can be presented as a dry product for constitution with water or other suitable vehicle before use. Preparations for oral administration can be suitably formulated to give controlled release of the active agent. For buccal administration the compositions can take the form of tablets or lozenges formulated in conventional manner.

For administration by inhalation, the agent for use according to the present invention is conveniently delivered in the form of an aerosol spray presentation from pressurized packs or a nebulizer, with the use of a suitable propellant, e.g., dichlorodifluoromethane, trichlorofluoromethaane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

An agent can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing or dispersing agents. Alternatively, the active agent can be in powder form for constitution with a suitable vehicle, e.g., sterile pyrogen-free water, before use.

In addition, an agent can also be formulated as a depot preparation. Such long acting formulations can be administered by implantation (for example, subcutaneously or intramuscularly) or by intramuscular injection. Thus, for example, an agent can be formulated with suitable polymeric or hydrophobic materials (for example as an emulsion in an acceptable oil) or ion exchange resins, or as sparingly soluble derivatives, for example, as a sparingly soluble salt.

An agent of the present invention can also be co-administered with another agent having similar biological activity. For example, the agent can be combined or otherwise co-administered with other therapeutics used in the treatment of heart failure, including diuretics, vasodilators and inotropic agents such as ACE inhibitors.

Toxicity and therapeutic efficacy of a selected agent can be determined by standard pharmaceutical procedures in cell cultures or experimental animals, e.g., for determining the LD₅₀ (the dose lethal to 50% of the population) and the ED₅₀ (the dose therapeutically effective in 50% of the population). For any agent used in the methods of the invention, the therapeutically effective dose can be estimated initially from animal models to achieve a concentration range that includes the IC₅₀ (i.e., the concentration of the test agent, which achieves half-maximal cardioprotection). Such information can be used to accurately determine useful doses in humans.

The invention is described in greater detail by the following non-limiting examples.

### Example 1

### Materials and Methods

Materials. Anti-Sir2α antibody, anti-acetyl-p53 (Lys 320 and Lys 373/382) antibodies and anti-acetyl histone H3 and H4 antibodies were purchased from Upstate. Sirtinol and DEVD-CHO were from CALBIOCHEM, and Trichostatin A (TSA) was from SIGMA.

Plasmids. A plasmid harboring the gene for mouse Sir2α is known in the art (Imai, et al. (2000) Nature 403:795-800). Dominant-negative Sir2α (DN-Sir2a) was generated by mutating histidine 355 to alanine (Luo, et al. (2001) Cell 107:137-148).

Primary Culture of Neonatal Rat Ventricular Myocytes. Primary cultures of cardiac ventricular myocytes were prepared from 1-day-old Crl: (WI) BR-Wistar rats according to standard methods (Tomita, et al. (2003) Circ. Res. 93:12-22). Myocytes were cultured under serum-free conditions for 48 hours before experiments. Cell size and total protein content were obtained (Tomita, et al. (2003) supra).

Immunostaining. Cells were fixed in PBS containing 3.7% paraformaldehyde, permeabilized in PBS containing 0.3% TRITON X, and blocked with 5% BSA. Immunostaining was performed using antimurine Sir2α antibody (1:100) and antisarcomeric myosin antibody (MF20; 1:500).

Assays for Apoptosis. Histone-associated DNA fragments were quantitated by the Cell Death Detection ELISA (ROCHE; Yamamoto, et al. (2003) J. Clin. Invest. 111:1463-1474).

Immunoblot Analysis. Nuclear fractions were prepared as described (Tomita, et al. (2003) supra). Whole-cell lysates were obtained using Lysis Buffer A, containing 150 mmol/L NaCl, 50 mmol/L Tris, pH 7.5, 0.1 mmol/L Na₃VO₄, 1 mmol/L NaF, 0.5 mmol 4-(2-aminoethyl)benzenesulfonyl fluoride hydrochloride, 1% NONIDET P-40, 0.1% sodium dodecyl sulfate, 0.5% deoxycholic acid, 0.5 μg/mL leupeptin, and 0.5 μg/mL aprotinin. For immunoblotting, the antimurine Sir2α antibody (1:10 000) was used.

Transfection and Luciferase Assays. One million cells were transfected with 4 μg of plasmid. The transcriptional activity of p53 was evaluated using p53-Luc (2 pg) containing 15 copies of specific p53 DNA-binding sequence (STRATAGENE). A plasmid carrying the β-galactosidase gene (SV40-β-gal; 1 μg) was cotransfected. One microgram of expression plasmids pcDNA3.1, pcDNA3.1-Sir2α, or pcDNA 3.1-DN-Sir2α was employed.

Adenoviral Vectors. Adenovirus harboring Sir2α (Ad-Sir2α) or DN-Sir2α (Ad-DN-Sir2a) was made using ADMAX (MICROBIX). Adenovirus harboring X-linked inhibitor of apoptosis (Ad-XIAP) and that harboring temperature sensitive dominant-negative p53 (Ad-DN-p53) have been described previously (Yamamato, et al. (2003) supra; Regula and Kirshenbaum (2001) J. Mol. Cell. Cardiol. 33:1435-1445).

Dog Model of Left Ventricular Hypertrophy and Heart Failure. Dog models of left ventricular hypertrophy (LVH) and LVH plus heart failure were prepared according to established methods (Hittinger, et al. (1989) Circ. Res. 65:971-980). Dogs were obtained from Marshall BioResources (North Rose, N.Y.).

Statistical Analyses. Data are given as mean±SEM. Statistical analyses were performed using ANOVA and post hoc tests by the Tukey method. Significance was accepted at the P<0.05 level.

What is claimed is:

1. A method for identifying a cardioprotective agent comprising contacting a cardiac cell with a test agent and determining whether the test agent modulates the expression or activity of Sir2α, wherein an increase in the expression or activity of Sir2α is indicative of a cardioprotective agent.

2. An agent identified by the method of claim 1.
